(19) United States
(12) Patent Application Publication
(10) Pub. No.: US 2024/0066209 A1
(43) Pub. Date: Feb. 29, 2024

(54) Dynamic Infusion Pump

(71) Applicant: Adrian Attard Trevisan, Balzan (MT)

(72) Inventor: Adrian Attard Trevisan, Balzan (MT)

(21) Appl. No.: 17/899,638

(22) Filed: Aug. 31, 2022

(51) Int. Cl. A61M 5/142; A61M 5/158

(52) U.S. Cl. CPC A61M 5/14248; A61M 5/158; A61M 2005/14252

(57) ABSTRACT

An improved system, method for an infusion pump is an electronic computerized device that allows to precisely time and dose intravenous or enteral therapy. The proposed infusion pump system increases patient comfort and convenience, while it is lightweight and modular. It comes with a user-friendly interface using onboard buttons and through an associated application, multiple types of controls can be practiced by the user through his/her smart device.

### BACKGROUND

### Field of the Invention

This invention relates to infusion pump systems and more particularly relates to an electronic computerized infusion pump that allows the user to precisely time and dose intravenous or enteral therapy.

### Description of the Related Art

Infusion pump designs were rarely seen in medical literature until the 1950s. Most of these early infusion pumps were extracorporeal devices of various designs. One such device included a reciprocating air pump driven by an electric motor. Yet another design considered comprised a metal housing for a glass syringe and a compression chamber fed by a tank of nitrogen gas. Yet another such infusion pump included a motorized syringe pump which included an electric motor connected to the worm drive that moved a syringe plunger by a gear box. The gears were interchangeable such that replacement of the gears permitted different delivery rates. Yet another infusion pump included a syringe plunger driven by a rider on a threaded shaft. While this is but a sampling of such devices, it should be appreciated that numerous other designs were considered and used for extracorporeal infusion devices.

Modern constant-flow implantable infusion devices, or implantable pumps, for delivering an infusate (e.g., medicaments, insulin, etc.) commonly have a rigid housing that maintains a collapsible infusate reservoir. The housing includes a needle-penetrable septum that covers a reservoir inlet. A flow passage is provided between the reservoir and an exterior surface of the device, such flow passage includes, or defines, a restrictor to establish a maximum output infusate flow rate. At the flow passage outlet, a flexible delivery catheter is provided.

Practically, such a device is implanted at a selected location in a body so that (i) the inlet septum is proximate to the patient's skin and (ii) a distal end of the catheter is positioned at a selected delivery site. Infusate can then be delivered to the infusion site by forcing such fluid from the device reservoir. When the infusate reservoir becomes empty, the reservoir is refillable through the septum inlet by injecting a new supply of infusate through the apparatus' inlet septum. Due to the location of the device in relation to the skin of the patient, the injection can be readily accomplished using a hypodermic needle (or cannula).

By looking at prior art there are multiple solutions that are presented bringing advancement in the chest tubes system. However, these solutions are limited and restricted to their conventional architecture, and installation system and have considerable shortcomings which adversely affect the convenience with which they can be used. The prior systems have certain limitations including the design of assemblies which make them not suitable for every type of patient. Moreover, the ease of use and delivery of medications is also questionable.

By looking at prior art, multiple advancements have been seen in similar regards. For instance, a U.S. Pat. No. 3,731,681A discusses an infusion pump which may be used to infuse fluids, medication, or nutrients into a patient's body or circulatory system. An infusion pump is generally used intravenously, although subcutaneous, arterial and epidural infusions are occasionally used. Infusion pumps can reliably administer fluids in ways that would be impractically expensive or unreliable if performed manually by nursing staff. For example, infusion pumps can administer 1 mL per hour injections (too small for a drip), injections every minute, injections with repeated boluses requested by the patient up to the maximum number per hour allowed (e.g. in patient-controlled analgesia), or fluids whose volumes vary by the time of day.

A U.S. Pat. No. 3,951,147A discusses an implantable infusate pump which is a rechargeable infusate pump for implantation in the human body can be refilled periodically by injection through an inlet septum under the skin. A conduit from the pump outlet conducts fluid to an infusion site in the body. The needle penetrating the septum protrudes into an antechamber and discharges fluid and any foreign matter is trapped in the antechamber so that it cannot enter the pump chamber and possibly clog the pump outlet. This foreign material is removed by injecting a needle into the antechamber and drawing a vacuum to suck out the debris. The pump also has a special ring-like outlet filter in the fluid stream between the pump chamber and its outlet to provide a maximum amount of filter area in a minimum amount of space and the pump outlet includes a special flow controller which is able to very accurately meter the infusate to the selected body site.

A WO patent 1,984,001718A1 on fluid handling system for medication infusion system discusses an apparatus (10) for fluid handling and delivery in a medication infusion system having a medication reservoir (14) for storing selected medication; a pump (52) for pumping the selected medication, whereby the pump (52) operates in a pulsatile mode capable of producing a pulsing output flow; an accumulator (36) for accumulating the selected medication, whereby the input of the accumulator (36) is in communication with the output of pump (52); a flow restrictor (72) for restricting the flow of the selected medication, whereby the flow restrictor (72) is in communication with the output accumulator (36), with the flow restrictor (72) in combination with the accumulator (36) smooth the pulsatile nature of the flow of selected medication; and a catheter (90) for communicating the selected medication from the output of the flow restrictor (72) to a living body.

An EP patent 0,450,186A1 discusses medication infusion device with dose recharge restriction which is a subcutaneously implantable medication infusion device includes a variable capacity reservoir (22) for receiving and storing fluid medication for delivery to a cathether (26) which directs the medication to a specific infusion location in the body. A control assembly (28) is interposed between the reservoir (22) and the catheter (26) to facilitate and control the transfer of the medication from the reservoir (22) to the catheter (26) in a safe and efficient manner. The control assembly (28) includes a self-recharging pump (50) and a normally closed valve (52), both of which are manually actuable by percutaneous pressure when subcutaneously implanted, and defines a portion of a fluid flow conduit (24) between the reservoir (22) and the catheter (26). The control assembly (28) is constructed to permit the infusion of a measured bolus of medication on demand through manual percutaneous manipulation of the control assembly (28) to limit the rate at which the pump (50) is recharged with medication, to restrict the total amount of medication which can be pumped into the catheter (26) over a given period of time. In one preferred form, the pump recharge restriction is created by providing at least one capillary-like fluid pathway (110) through which the recharge fluid must pass before entering the pump (50). In another preferred form of the invention, the recharge flow rate of medication into the pump (50) is restricted by a wick restrictor (118).

A U.S. Pat. No. 7,914,499B2 discusses a multi-cartridge fluid delivery device which is a fluid delivery device for administering a first medicament and a second medicament includes a first fluid reservoir configured to contain the first medicament and a second fluid reservoir configured to contain the second medicament. The fluid delivery device may include one or more basal drive mechanisms to provide a basal delivery of one or more of the first and second medicaments. The fluid delivery device may further include one or more bolus drive mechanisms to provide a bolus delivery of one or more of the first and second medicaments.

There are multiple solutions that have been presented in prior art providing advancements in their own domain. However, these solutions are limited and restricted to their conventional architecture, and installation system and have considerable shortcomings which adversely affect the convenience with which they can be used. The prior systems have certain limitations including the design of assemblies which make them not suitable for every type of use. Moreover, the ease-of-use ability of these assemblies is also questionable. The current invention proposes an advanced methodology of infusion pump which is an electronic computerized device that allows the health care staff to precisely time and dose intravenous or enteral therapy. Therapy can be continuous, meaning it is given continuously for 24 hours, or intermittent, meaning it is given at specific times and for a short period of time. Drugs, liquids, enteral nutrition, and parenteral nutrition can all be administered with the help of infusion pumps.

None of the previous inventions and patents, taken either singly or in combination, is seen to describe the instant invention as claimed. Hence, the inventor of the present invention proposes to resolve and surmount existent technical difficulties to eliminate the aforementioned shortcomings of the prior art.

### SUMMARY

In light of the disadvantages of the prior art, the following summary is provided to facilitate an understanding of some of the innovative features unique to the present invention and is not intended to be a full description. A full appreciation of the various aspects of the invention can be gained by taking the entire specification, claims, drawings, and abstract as a whole.

The primary desirable object of the present invention is to provide a novel and improved form of an infusion pump that is electronic in nature and requires minimal input from the health care staff.

Another object of the present invention is to provide a novel device that is lightweight, and modular in nature.

It is a more particular object of the present: invention to provide an improved system that assists doctors in precisely controlling the medications.

It is also an object of the invention to provide an assembly that comes with a user-friendly interface local using onboard buttons and through 3rd BT/BLE enabled device running Android/iOS App via internal BLE module which enables customization of various settings incl. flow rates and volumes as desired by the user according to the application.

It is also the objective of the invention to provide an advanced approach where the actuation mechanism is composed of a Stepper Motor with high-resolution micro-stepping drive circuitry to enable high flow accuracy.

It is also the objective of the invention to provide an assembly that is designed to be flexible and easy to use. Furthermore, it is intended to be small, lightweight, and modular.

It is also the objective of the invention to provide a whole construction that is compatible with high-volume manufacturing methods for all of the components. The low manufacturing cost means that this high-quality portable instrument may be made available to hospitals and clinics that may otherwise not be able to afford a quality instrument. It also makes the assembly highly applicable to the developing world and exposes a wider public audience to first-hand scientific advancements.

Thus, it is the objective to provide a new and improved form of the electronic computerized infusion pump. Other aspects, advantages, and novel features of the present invention will become apparent from the detailed description of the invention when considered in conjunction with the accompanying drawings.

This Summary is provided merely for the purposes of summarizing some example embodiments, so as to provide a basic understanding of some aspects of the subject matter described herein. Accordingly, it will be appreciated that the above-described features are merely examples and should not be construed to narrow the scope or spirit of the subject matter described herein in any way. Other features, aspects, and advantages of the subject matter described herein will become apparent from the following Detailed Description, Figures, and Claims.

### BRIEF DESCRIPTION OF THE DRAWINGS

The accompanying figures, where like reference numerals refer to identical or functionally similar elements throughout the separate views, together with the detailed description below, are incorporated in and form part of the specification, and serve to illustrate further embodiments of concepts that include the claimed invention and explain various principles and advantages of those embodiments.

FIG. 1 is a perspective view of an electronic computerized infusion pump showing my new design;

FIG. 2 is another perspective view of an electronic computerized infusion pump;

FIG. 3 shows the perspective use of an electronic computerized infusion pump.

Skilled artisans will appreciate that elements in the figures are illustrated for simplicity and clarity and have not necessarily been drawn to scale. For example, the dimensions of some of the elements in the figures may be exaggerated relative to other elements to help to improve understanding of embodiments of the present invention.

The apparatus and method components have been represented where appropriate by conventional symbols in the drawings, showing only those specific details that are pertinent to understanding the embodiments of the present invention so as not to obscure the disclosure with details that will be readily apparent to those of ordinary skill in the art having the benefit of the description herein.

### DETAILED DESCRIPTION

Detailed descriptions of the preferred embodiment are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in virtually any appropriately detailed system, structure or manner.

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

An infusion pump may be used to infuse fluids, medication, or nutrients into a patient's body or circulatory system. An infusion pump is generally used intravenously, although subcutaneous, arterial and epidural infusions are occasionally used. Infusion pumps can reliably administer fluids in ways that would be impractically expensive or unreliable if performed manually by nursing staff. For example, infusion pumps can administer 1 mL per hour injections (too small for a drip), injections every minute, injections with repeated boluses requested by the patient up to the maximum number per hour allowed (e.g. in patient-controlled analgesia), or fluids whose volumes vary by the time of day.

In one embodiment, an enhanced structure of an infusion pump is disclosed which is electrical in nature and can be controlled through touch screen buttons or through an associated application.

In another embodiment, Infusion pumps are positive pressure pumps capable of controlling the drops that are infused. The device as per its additional embodiments can be used for various surgical purposes. It can be used for including but not limited to which are commonly used for the treatment of Parkinson's disease, Palliative Care, Thalassemia, Subcutaneous Immunoglobulins and Pulmonary Arterial Hypertension.

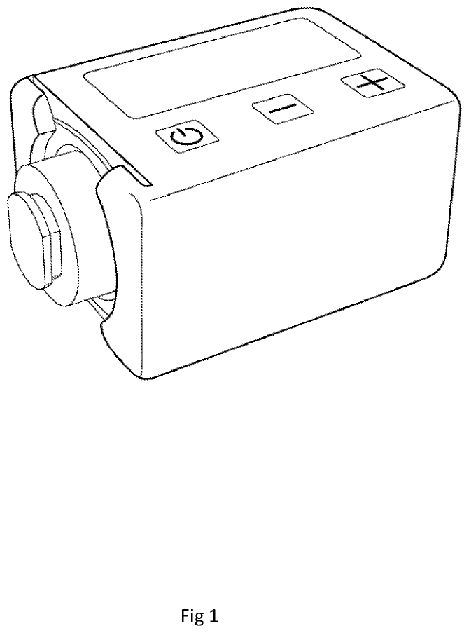

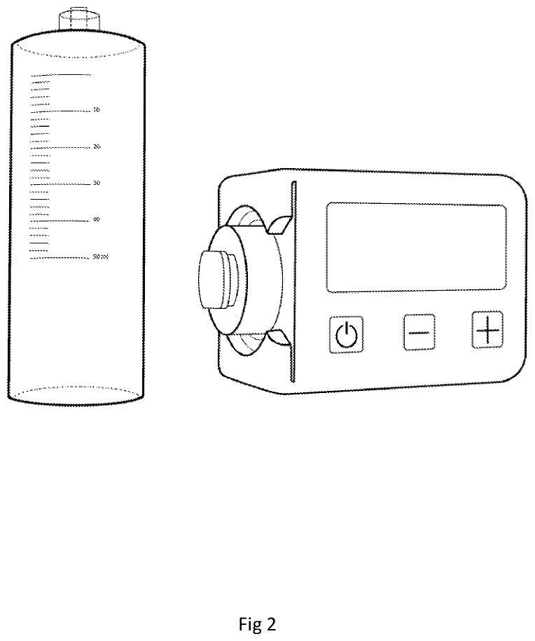

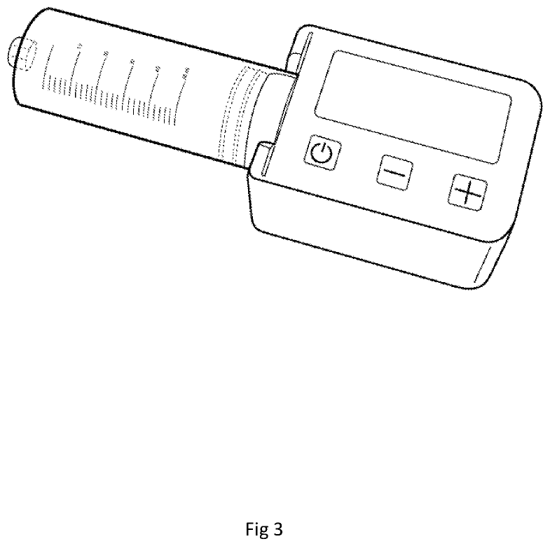

By looking at FIG. 1-FIG. 3 the structure of the assembly can be seen. FIG. 1 is a perspective view of an electronic computerized infusion pump and FIGS. 2 and 3 are showing the infusion pump ready to be used. The configuration of assembly allows to control of dosage and flow rate at specific times and for a short period of time through a controlled interface.

The assembly as per its further embodiments presents the following features. The single-use reservoir capacity of assembly depends on the specific syringe used (10 ml-50 ml). The volumes that can be administered are selectable from 1 ml to the capacity of the syringe used in increments of 1 ml. The flow rates and delivery time are programmable in nature.

The Bolus Dose Volume is programmable from 0 ml to 2 ml in increments of 0.02 ml. The Flow Rate Accuracy is ±4% and Occlusion Pressure is a selectable Occlusion Pressure value. The Post Occlusion Bolus is around 1.2 ml.

The internal electronics of assembly involve Electronic Circuitry which is 32 bit Processor/BLE 5.0 (Bluetooth connectivity). The settings can be set locally or received from BLE and are automatically stored inside Flash Memory. The display of the device is OLED Display 128×64 Pixels. The assembly involves a high-resolution stepper motor and alarms in case of detection of any abnormalities, show messages and errors on display and transmit them via BLE.

While a specific embodiment has been shown and described, many variations are possible. With time, additional features may be employed. The particular shape or configuration of the platform or the interior configuration may be changed to suit the system or equipment with which it is used.

Having described the invention in detail, those skilled in the art will appreciate that modifications may be made to the invention without departing from its spirit. Therefore, it is not intended that the scope of the invention be limited to the specific embodiment illustrated and described. Rather, it is intended that the scope of this invention be determined by the appended claims and their equivalents.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separately claimed subject matter.

We claim:

1. A flexible and easy-to-use infusion pump assembly wherein the assembly comprises of:
an infusion site interface structure that is adhesively attachable to a skin surface, the infusion site interface structure comprising: a frame with one surface facing toward the skin surface and an upward facing surface that is opposite the lower surface, a soft cannula coupled to the frame and extending below the lower surface of the frame for insertion subcutaneously through the skin surface to at least partially define a fluid path, and a septum arranged in the fluid path and coupled to the frame in a position above the upward facing surface of the frame;
multiple options are given on board; and,
an associated application that can run on smart devices for ease of use.
As per claim 1, the assembly is an electronic infusion pump computerized device allowing to precisely time and dose intravenous or enteral therapy;
As per claim 1, the assembly is small, portable, and modular in nature;
As per claim 1, the assembly provides various customization settings through on-board buttons;
As per claim 1, the assembly provides various customization settings through the associated application; and
As per claim 1, the assembly's actuation mechanism is composed of a Stepper Motor with a high-resolution micro-stepping drive circuitry to enable high flow accuracy.
